(19) United States
(12) Patent Application Publication
(10) Pub. No.: US 2010/0048898 A1
(43) Pub. Date: Feb. 25, 2010

(54) Process for Synthesizing 7-Alkynyl-4- Aminoquinazolines and a Related Intermediate

(76) Inventor: Eric N. Jacobsen, Boston, MA (US)

Correspondence Address: Henry D Coleman;Coleman Sudol Sapone, 714 Colorado Avenue, Bridgeport, CT, 06605, US

(21) Appl. No.: 12/224,412

(22) PCT Filed: Mar. 2, 2007
(86) PCT No.: PCT/US2007/005468

§ 371 Date: Nov. 17, 2008

Related U.S. Application Data
(60) Provisional application No. 60/778,805, filed on Mar. 3, 2006.

(51) Int. Cl. C07D 239/72

(52) U.S. Cl. 544/287

(57) ABSTRACT

A process for synthesizing 7-alkynyl-4-aminoquinazolines from 7-haloquinazolines is disclosed. In one specific synthesis, 4-[N-3-chloro-4-fluorophenyl)]-7-[3-methyl-3-(4-methyl-1-piperazinyl)-1-butynyl]-6-[N-methyl acrylamide]quinazoline is prepared from 7-chloro-6-nitro-3H-quinazolin-4-one. Also disclosed is an intermediate useful in the syntheses of 7-alkynyl-4-ammoquinaozolines and a process for making the intermediate. The 7-alkynyl-4-aminoquinaozolines prepared by processes of the invention are useful as pharmaceutically active compounds.

### RELATED APPLICATIONS

This application claims the benefit of priority of provisional application U.S. 60/778,805 of identical title, filed Mar. 3, 2006.

### FIELD OF THE INVENTION

The field of the invention is synthetic organic chemistry, more particularly, pharmaceutical chemistry and quinazoline derivatives.

### BACKGROUND OF THE INVENTION

HER2, ErbB3, and ErbB4 belong to the ErbB family and form a heterocomplex that interacts in intracellular signal transduction. Co-expression of the EGF receptor and HER2 accelerates tumorigenesis, is associated with poor prognoses in breast cancer, oral cancer, and lung cancer, and is associated with resistance to endocrine therapy in breast cancer.

Certain 4-aminoquinazolines inhibit EGF receptor tyrosine kinase and HER2 tyrosine kinase and may prove useful in the treatment of a wide variety of cancers. United States Patent Publication No. 2004/0116422 (the complete disclosure of which is hereby incorporated by reference) discloses syntheses of 7-alkynyl-4-aminoquinaozolines which are useful inhibitors of EGF receptor tyrosine kinase and HER2 tyrosine kinase. PCT WO2005/051924 also describes syntheses of 7-alkynylaminoquinaozolines.

Useful anticancer agents disclosed in United States Patent Publication No. 2004/0116422 include 4-[N-3-chloro-4-fluorophenyl)]-7-[3-methyl-3-(4-methyl-1-piperazinyl)-1-butynyl]-6-[N-methyl acrylamide]quinazoline, which has the following structure:

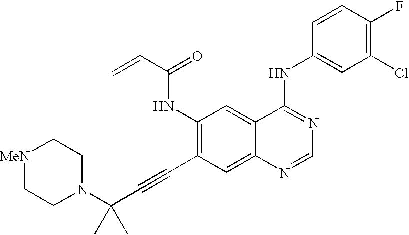

The syntheses described in United States Patent Publication No. 2004/0116422 substitute the 7-position of a 4-aminoquinazoline with an acetylinic moiety. These syntheses would be simplified—and process efficiencies enhanced—if the acetylenic moiety could be introduced prior to formation of the 4-aminoquinazoline pharmacore, thereby reducing the requisite number of process steps.

Accordingly, the need exists for new and improved processes for making 7-alkynyl-4-aminoquinaozoline anti cancer agents.

### SUMMARY OF THE INVENTION

The invention provides improved and readily-scalable processes for synthesizing 7-alkynyl-4-aminoquinaozolines in high yields. Processes of the invention minimize the number of process steps needed to make 7-alkynyl-4-aminoquinaozolines and achieve high yields of purified products.

In one embodiment, the invention provides a process for synthesizing 7-alkynyl-4-aminoquinazoline compounds having the formula (1):

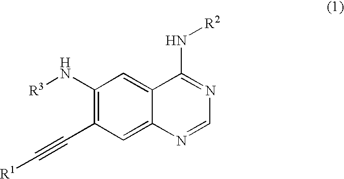

in which R¹ is:

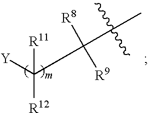

where (i) R⁸ and R⁹ are each independently a hydrogen atom, or (ii) R⁸ and R⁹ are each independently a C₁-C₅ alkyl group optionally substituted by a C₁-C₅ alkoxy group, m is an integer of 0-3, R¹¹ and R¹² are each independently a hydrogen atom or a C₁-C₅ alkyl group, and Y is a hydrogen atom, a hydroxyl group, a C₁-C₅ alkoxy group, a C₁-C₅ alkanoyloxy group, 4-C₁-C₅ alkylpiperazin-1-yl, di(C₁-C₅ alkyl)amino, —N(R¹⁶)—(CO)ᵤ—(CR¹⁷R¹⁸)ᵥ—(CO)ⱼ—R¹⁹ (wherein R¹⁶ is a hydrogen atom, or a C₁-C₅ alkyl group optionally substituted by a cyano group or a C₁-C₅ alkoxy group, R¹⁷ and R¹⁸ are each independently a hydrogen atom or a C₁-C₅ alkyl group, u and j are each 0 or 1, v is an integer of 1-5 and R¹⁹ is a hydrogen atom, a hydroxyl group, a cyano group, an amino group, a C₁-C₅ alkoxy group, a morpholino group, 4-C₁-C₅ alkylpiperazin-1-yl or di(C₁-C₅ alkyl) amino;
in which R³ is:

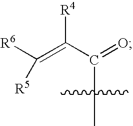

and R⁴, R⁵, and R⁶ are each independently a hydrogen atom, a halogen atom (F, Cl, Br, I) or a C₁-C₅ alkyl group optionally substituted by a halogen atom, a morpholino group, 4-C₁-C₅ alkylpiperazin-1-yl or di(C₁-C₅ alkyl)amino; and
in which R² is:

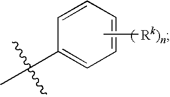

where n is an integer of 0-3 and R^{k} is a hydrogen atom, a halogen atom, a hydroxyl group, a cyano group, a nitro group, a trifluoromethyl group, a C₁-C₅ alkyl group, a C₁-C₅ alkoxy group, —S(O)_{f}R¹³ (wherein f is an integer of 0-2 and R¹³ is a C₁-C₅ alkyl group), —NR¹⁴R¹⁵ (wherein R¹⁴ and R¹⁵ are each independently a hydrogen atom, a C₁-C₅ alkyl group, a C₁-C₅ alkanoyl group, or a C₁-C₅ alkylsulfonyl group, a C₁-C₅ alkenyl group, a C₁-C₅ alkynyl group, or a C₁-C₅ alkanoyl group,
the process comprising:
(a) derivatizing 7-chloro-6-nitro-3H-quinazolin-4-one at the 7-position with a moiety of formula (IA):

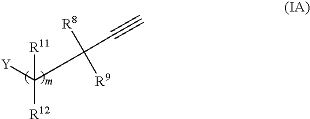

to form a first reaction product, where R⁸, R⁹, R¹¹, R¹², Y, and m are as defined above, by reacting—through Sonogashira coupling (see, e.g., Sonogashira et al., Tetrahedron Lett., 1975, 4467) or other reaction conditions or mechanisms which favor substitution of the 7-position chlorine of 7-chloro-6-nitro-3H-quinazolin-4-one with an acetylenic moiety (e.g., the conditions specified in Examples 1 and 2 herein)—approximately equimolar amounts of 7-chloro-6-nitro-3H-quinazolin-4-one and a compound of formula (IA) at a temperature of between about 20° C. to about 100° C.;
(b) reacting the first reaction product with an approximately equimolar amount of a compound of the formula (NH₂)—R² in a reaction medium comprising an halogenating agent (e.g., SOCl₂) and at a temperature of between about 0° C. to about 100° C. to form a derivatized 7-alkynyl4-aminoquinazoline as a second reaction product, where R² is as defined above;
(c) reducing the nitro group of the second reaction product thereby forming a third reaction product under reaction conditions that are compatible with the acetylenic and haloaromatic functionalities in the second and third reaction products, e.g., by reacting the second reaction product under reducing conditions for example, with a reducing agent such as hydrazine and a Lewis acid such as FeCl₃ at a temperature of between about 20° C. to about 100° C. at an approximately 2:1 molar ratio of hydrazine:second reaction product; and
(d) reacting the third reaction product at a temperature of between about 0° C. to about 50° C. with an approximately equimolar amount of an acylating agent, such as an acylating agent derived by combining acrylic acid and acryloyl chloride (ACC). Other reducing conditions which can be used to reducing the nitro group to an amine group as described above may include one or more of Zn/water, Zn/HCl, Zn/NaOH, Zn/NH₃, Fe, Fe/HOAC, Fe/HCl, Sn/HCl, SnCl₂/HCl, Na₂S, Na₂S₂, Na₂S₂O₄, (NH₄)₂S, NaBH₄/CuCl, Al₂Te₃/H₂, PhNHNH₂ (phenylhydrazine), NaO₂CH/KH₂PO₄, CO/H₂O/Se/Et₃N, Fe(CO)₅, Fe₃(CO)₁₂/Al₂O₃. One of ordinary skill in the art will recognize that reducing conditions may be applied or chosen from analogous literature preparations so that a high yield of the amine reduction product (from the nitro group reduction) will occur while minimizing any unfavorable reactions with other functional groups in the molecule.

Processes of the invention are conducted at approximately atmospheric pressure and can be done one-pot or in steps using reactant amounts and reaction media which are either described herein or which can be determined by those of ordinary skill in the art.

In one embodiment, the invention provides a process for synthesizing 4-[N-3-chloro-4-fluorophenyl)]-7-[3-methyl-3-(4methyl-1-piperazinyl)-1-butynyl]-6-[N-methyl acrylamide]quinazoline by:
(a) reacting 7-[3-methyl-3-(4-methyl-piperazin-1-yl)but-1-ynyl]-6-nitro-quinazolin-4-ol (A) with an approximately equimolar amount of 3-chloro-4-fluoro-phenylamine (CFA) at a temperature of between about 0° C. to about 100° C. and in a reaction medium comprising a halogenating agent (e.g., thionyl halide or phosphorous oxychloride or another halogenating agent as defined herein) to form 4-[N-3-chloro-4-fluorophenyl)]-7-[3-methyl-3-(4-methyl-1-piperazinyl)-1-butynyl]-6-[nitro]quinazoline (B);
(b) reacting 4-[N-3-chloro-4-fluorophenyl)]-7-[3-methyl-3-(4-methyl-1-piperazinyl)-1-butynyl]-6-[nitro]quinazoline (B) under reducing conditions, e.g. with hydrazine at an approximately 2:1 molar ratio of hydrazine: 4-[N-3-chloro-4-fluorophenyl)]-7-[3-methyl-3-(4methyl-1-piperazinyl)-1-butynyl]-6-[nitro]quinazoline, at a temperature of between about 20° C. to about 100° C. and in a reaction medium comprising a Lewis acid (e.g., FeCl₃), thereby forming 4-[N-3-chloro-4-fluorophenyl)]-7-[3-methyl-3-(4-methyl-1-piperazinyl)-1-butynyl]-6-[amino]quinazoline (C); and
(c) reacting 4-[N-3-chloro-4-fluorophenyl)]-7-[3-methyl-3-(4-methyl-1-piperazinyl-1-butynyl]-6-[amino]quinazoline (C) with an approximately equimolar amount of acrylic acid and acryloyl chloride (ACC) at a temperature of between about 0° C. to about 50° and in a reaction medium comprising triethyl amine or other amine acid scavenger (an amine acid scavenger is an amine compound which complexes with acid produced during a reaction to form an amine acid salt and render the acid inert but does not otherwise participate in a reaction). Examples of amine acid scavengers include triethylamine and pyridine, among others.

In a preferred embodiment, the invention provides a process for synthesizing a pharmaceutically acceptable salt (e.g., a tosic acid salt) of 4-[N-3-chloro-4-fluorophenyl)]-7-[3-methyl-3-(4-methyl-1-piperazinyl)-1-butynyl]-6-[N-methyl acrylamide]quinazoline by:
(a) reacting 7-[3-methyl-3-(4-methyl-piperazin-1-yl)-but-1-ynyl]-6-nitro-quinazolin-4-ol (A) with an approximately equimolar amount of a N-protected 3-chloro-4-fluoro-phenylamine derivative, e.g., Boc-3-chloro-4-fluoro-phenylamine derivative (Boc-CFA), at a temperature of between about 0° C. to about 100° C. and in a reaction medium comprising a halogenating agent (e.g., thionyl halide or phosphorous oxychloride or another halogenating agent as defined herein) and a base, preferably a strong base such as NaH (sodium hydride—other bases include for example, alkyl lithium, such as butyl lithium, or phenyl lithium, lithium di-alkylamide, for example, lithium diisopropylamide, lithium amide, tertiary potassium butylate, sodium amide, sodium t-butoxide, potassium t-butoxide, LiN(SiMe₃)₂, among numerous other bases including NaOH, K₂CO₃, Na₂CO₃, etc.) to form a 4-[N-protected 3-chloro-4-fluorophenyl)]-7-[3-methyl-3-(4-methyl-1-piperazinyl)-1-butynyl]-6-[nitro]quinazoline, e.g., 4-[N-Boc 3-chloro-4-fluorophenyl)]-7-[3-methyl-3-(4-methyl-1-piperzinyl)-1-butynyl]-6-[nitro]quinazoline (B¹);
(b) reacting the 4-[N-protected 3-chloro-4-fluorophenyl)]-7-[3-methyl-3-(4-methyl-1-piperazinyl)-1-butynyl]-6-[nitro]quinazoline, e.g., 4-[N-Boc-3-chloro-4-fluorophenyl)]-7-[3-methyl-3-(4-methyl-1-piperazinyl)-1-butynyl]-6-[nitro]quinazoline (B¹), with hydrazine at an approximately 2:1 molar ratio of hydrazine: 4-[N-protected-3-chloro-4-fluorophenyl)]-7-[3-methyl-3-(4-methyl-1-piperazinyl)-1-butynyl]-6-[nitro]quinazoline, at a temperature of between about 20° C. to about 100° C. and in a reaction medium comprising a Lewis acid (e.g., FeCl₃), thereby forming a 4-[N-protected-3-chloro-4-fluorophenyl)]-7-[3-methyl-3-(4-methyl-1-piperazinyl)-1-butynyl]-6-[amino]quinazoline, e.g., 4-[N-Boc-3-chloro-4-fluorophenyl)]-7-[3-methyl-3-(4-methyl-1-piperazinyl)-1-butynyl]-6-[amino]quinazoline (C¹);
(c) reacting the 4-[N-protected-3-chloro-4-fluorophenyl)]-7-[3-methyl-3-(4-methyl-1-piperazinyl)-1-butynyl]-6-[amino]quinazoline, e.g., 4-[N-Boc-3-chloro-4-fluorophenyl)]-7-[3-methyl-3-(4-methyl-1-piperazinyl)-1-butynyl]-6-[amino]quinazoline (C¹), with an approximately equimolar amount of acrylic acid and acryloyl chloride (ACC) at a temperature of between about 0° C. to about 50° and in a reaction medium comprising an amine acid scavenger such as triethyl amine to form a 4-[N-protected-3-chloro-4-fluorophenyl)]-7-[3-methyl-3-(4-methyl-1-piperazinyl)-1-butynyl]-6-[N-methyl acrylamide]quinazoline, e.g., 4-[N-Boc-3-chloro-4-fluorophenyl)]-7-[3-methyl-3-(4-methyl-1-piperazinyl)-1-butynyl]-6-[N-methyl acrylamide]quinazoline (D¹); and
(d) deprotecting the 4-[N-protected-3-chloro-4-fluorophenyl)]-7-[3-methyl-3-(4-methyl-1-piperazinyl)-1-butynyl]-6-[N-methyl acrylamide]quinazoline, e.g., 4-[N-Boc-3-chloro-4-fluorophenyl)]-7-[3-methyl-3-(4-methyl-1-piperazinyl)-1-butynyl]-6-[N-methyl acrylamide]quinazoline (D¹), under conditions to remove the protecting group, for example (as in the case of the Boc group), in an acidic reaction medium comprised of an alcohol, e.g., an acidic reaction medium comprised of TsOH and methanol.

An example of the aforementioned preferred embodiment is illustrated by the following reaction schemes:

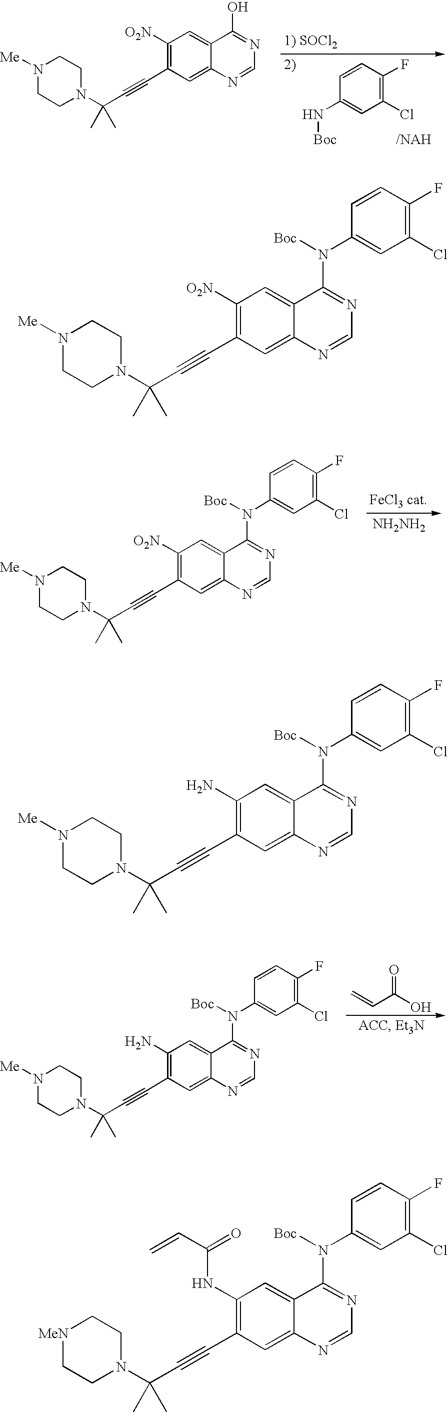

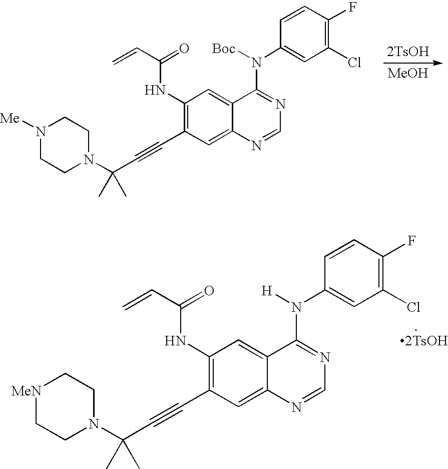

Step (a) of the preferred embodiment described above can use CFA derivatives in which the CFA amine group of is protected by a variety of protecting groups, including acid-removable (acid-labile) protecting groups, such as a t-butyloxycarbonyl group, n-butyloxycarbonyl group other substituted oxycarbonyl (e.g., N-alphafluorenyloxycarbonyl, hexadienyloxycarbonyl) group, various carbamate groups such as methyl or ethyl carbamate (deprotection under a variety of conditions), 2,2,2-trichloroethylcarbamate or Troc group (deprotection with a reducing agent such as Zn, in water at a pH of about 4.2), or a trityl group such as a methyltrityl group or methoxytrityl group, among others. An exhaustive listing of alternative protecting groups which may be used in the present invention may be found in the text “Protecting Groups in Organic Synthesis, 3^{rd} Edition”, by Philip J. Kocienski or “Greene's Protective Groups in Organic Synthesis, 4^{th} Edition” by Peter G. M. Wuts and Theodora W. Greene. Steps (b)-(d) of the preferred embodiment described above proceed in the same manner irrespective of which protecting group is used, the only difference being the modification of conditions during the removal of the protecting group, which must be effective to remove the protecting group without appreciably impacting other groups within the molecule.

As illustrated in the reaction scheme below, 7-[3-methyl-3-(4methyl-piperazin-1-yl)-but-1-ynyl]-6-nitro-quinazolin-4-ol (A) can be formed by derivatizing 7-chloro-6-nitro-3H-quinazolin-4-one (3) at the 7-position with 3-methyl-3-(4-methyl-piperazin-1-yl)-but-1-ynyl (PBN). In an exemplary reaction, this occurs using approximately equimolar amounts of 1-(1,1-dimethyl-prop-2-ynyl)-4-methyl-piperazine and 7-chloro-6-nitro-3H-quinazolin-4-one at a temperature of between about 20° C. to about 100° C. and in a reaction medium comprising an amine acid scavenger such as triethyl amine, a polar aprotic solvent (e.g., dimethylacetamide (DMA), dimethylformamide (DMF), acetonitrile, or dimethyl sulfoxide, most preferably dimethyl sulfoxide) and a palladium catalyst (most preferably, a palladium chloride catalyst).

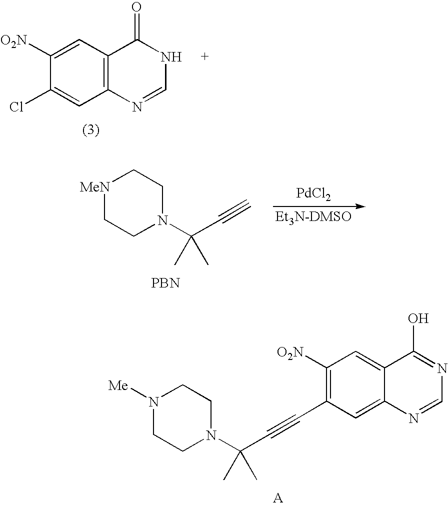

Alternatively, as shown in the following reaction, 7-[3-methyl-3-(4-methyl-piperazin-1-yl)-but-1-ynyl]-6-nitro-quinazolin-4-ol (A) can be formed by derivatizing 7-chloro-6-nitro-3H-quinazolin-4-one (3) at the 7-position with 3-methyl-3-(4-methyl-piperazin-1-yl)-but-1-ynyl (PBN) by reaction of approximately equimolar amounts of 1-(1,1-dimethyl-prop-2-ynyl)-4-methyl-piperazine and 7-chloro-6-nitro-3H-quinazolin-4-one at a temperature of between about 20° C. to about 100° C. or above and in a reaction medium comprising a solvent, preferably a polar aprotic solvent such as DMA, DMF, DMSO, acetonitrile, etc.

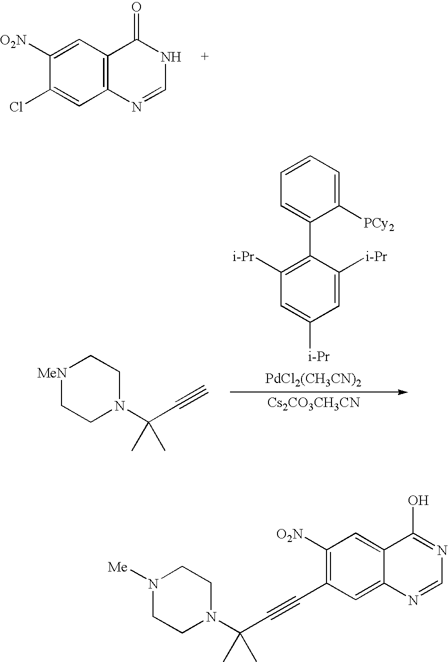

In another embodiment, the invention provides the compound 7-[3-methyl-3-(4-methyl-piperazin-1-yl)-but-1-ynyl]-6-nitro-quinazolin-4-ol (A):

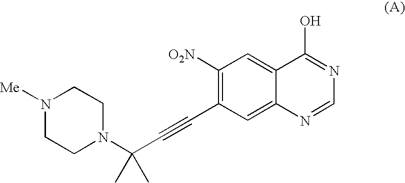

which, as described above, is useful as an intermediate in the synthesis of 7-alkynyl-4-aminoquinazolines.

In still another embodiment, the invention provides a process comprising derivatizing 7-chloro-6-nitro-3H-quinazolin-4-one (3)

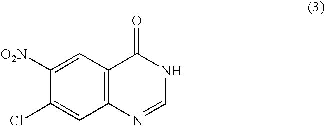

at the 7-position with a moiety of formula (IA) by reacting—through Sonogashira coupling or other reaction mechanism which favors substitution of the 7-position chlorine of 7-chloro-6-nitro-3H-quinazolin-4-one with an acetylenic moiety—approximately equimolar amounts of 7-chloro-6-nitro-3H-quinazolin-4-one and a compound of formula (IA) at a temperature of between about 20° C. to abut 100° C.

In still another embodiment the invention provides a process for synthesizing 7-[3-methyl-3-(4-methyl-piperazin-1-yl)-but-1-ynyl]-6-nitro-quinazolin-4-ol (A) by reacting 7-chloro-6-nitro-3H-quinazolin-4-one (3) and 1-(1,1-dimethyl-prop-2-ynyl)-4-methyl-piperazine (PBN) under reaction conditions which favor substitution of the 7-position chlorine of 7-chloro-6-nitro-3H-quinazolin-4-one. For example, 7-chloro-6-nitro-3H-quinazolin-4-one (3) and 1-(1,1-dimethyl-prop-2-ynyl)-4-methyl-piperazine (PBN) are reacted in a reaction medium comprising triethyl amine or other amine acid scavenger, a polar aprotic solvent (e.g., dimethylacetamide (DMA), dimethylformamide (DMF), acetonitrile, or dimethyl sulfoxide, most preferably dimethyl sulfoxide), and a palladium catalyst (preferably a palladium chloride catalyst) in accordance with the following reaction scheme:

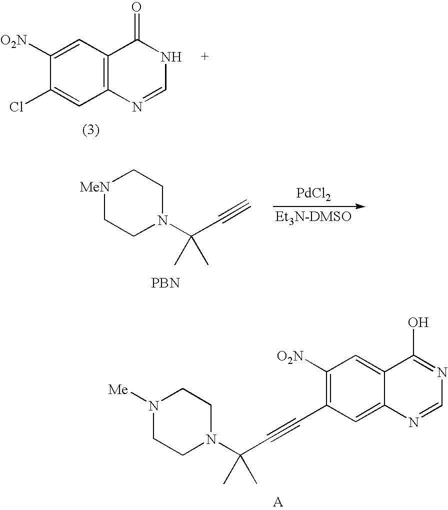

These and other aspects of the invention are described further in the following detailed description.

### DETAILED DESCRIPTION OF THE INVENTION

The following definitions apply unless indicated otherwise.

The term “compound”, as used herein, unless otherwise indicated within context, refers to any specific chemical compound(s) disclosed herein. The compounds of the present invention include all stereoisomers (i.e, cis and trans isomers), tautomers, and all optical isomers of the present compound and related analogs within context (eg., R and S enantiomers), as well as racemic, diastereomeric and other mixtures of such isomers, as well as all polymorphs and salts of the compounds, where relevant.

The term “protecting group” refers to a group which renders a functional group in a molecule such as an amine group inert to further reaction conditions and can be readily removed under conditions which do not appreciably impact the rest of the molecule. “Acid-removable protecting groups” are protecting groups as defined above which are removed under acidic conditions.

The term “alkyl” as used herein, unless otherwise indicated, includes saturated and monovalent C₁ to C₇ hydrocarbon radicals having straight, branched, or cyclic moieties or combinations thereof. C₁ to C₅ alkyls are preferred.

“Alkenyl” means a branched or unbranched hydrocarbon group containing 2 to about 7 carbon atoms and at least one double bond, such as ethenyl, n-propenyl, isopropenyl, n-butenyl and isobutenyl. C₁ to C₅ alkenyls are preferred.

“Alkynyl” means a branched or unbranched hydrocarbon group containing 2 to about 7 carbon atoms and at least one triple bond, such as ethynyl, n-propynyl, isopropynyl, n-butynyl, and isobutynyl. C₁ to C₅ alkynyls are preferred.

“Alkoxy groups” include but are not limited to an alkyl group bound through an ether linkage; that is, an “alkoxy” group may be represented as —O-alkyl where alkyl is as defined above. C₁ to C₅ alkoxy groups are preferred.

“Alkanoyl groups” means an acyl group derived from an alkanecarboxylic acid such as acetyl, propionyl, and butyryl, among others.

“4-C₁-C₅ alkylpiperazin-1-yl” means a piperazin-1-yl group substituted at the 4-position with alkyl as defined herein. 4methyl-piperazin-1-yl is preferred.

“Halogenating agents” are agents which introduce a halogen atom into a compound and include, but are not limited, to thionyl halides (preferably thionyl chloride) or phosphorous oxychloride, as well as a phosphorus halide (e.g., phosphorus trichloride, phosphorus tribromide, phosphorus pentachloride), and hydrochloric acid.

“Reducing agents” and “base” include but are not limited to hydrazine, as well as hydrogen gas (generally with a metal catalyst such as Pt or Pd), phenylhydrazine, Sn/HCl, SnCl₂/HCl, Zn/H₂O, NaBH₄/CuCl, NaBH₄/TiCl₄, Fe, Na₂S, NaH, LiH, and KH, as well as other examples of same as are otherwise set forth herein.

“Lewis acids” include but are not limited to FeCl₃, aluminum chloride, boron trifluoride, niobium pentachloride, and ytterbium (III) triflate.

“Bases”, especially including “strong bases” are those described above. These generally facilitate reactions by abstracting protons from a weak acid, thus producing strongly nucleophilic species which can participate in reactions.

The term “N-protected”, when used in chemical names such as “4-[N-protected 3-chloro-4-fluorophenyl)]-7-[3-methyl-3-(4-methyl-1-piperazinyl)-1-butynyl]-6-[nitro]quinazoline”, means that the nitrogen atom substituent, for example, at the quinazoline 4-position, is protected by a removable, preferably an acid-removable protecting group as defined herein.

“Reacting the second reaction product with a reducing agent and a Lewis acid” entails reducing the nitro group of the second reaction product, thereby forming a third reaction product under reaction conditions that are compatible with the acetylenic and haloaromatic functionalities in the second and third reaction products, i.e., reacting the second reaction product with a reducing agent and a Lewis acid (e.g. FeCl₃, BF₃) or an appropriate heterogeneous or homogeneous catalyst (e.g., Pd, Ni, Pt, Ru, or Rh-based catalysts) under reaction conditions which favor the selective reduction of aromatic nitro compounds to aromatic amines. Preferably, hydrazine is the reducing agent and FeCl₃ is the Lewis acid. A number of other approaches may be used as well to reduce the aromatic nitro group without affecting other functional groups in the molecule.

“Acylating agents” include an acylating agent derived by the combination of acrylic acid or, more generally, a carboxylic acid, with an activating agent such as acryloyl chloride (ACC), carbodiimide reagents (e.g. dicyclohexylcarbodiimide, diisopropylcarbodimide), EDC, HOBt, HATU, BOP, pyBOP, or any of the other known activating agents. Alternatively, carboxylic acid derivatives such as acid halides (esp. acid chlorides), thioesters, anhydrides, or esters can be used as acylating agents either in the absence or presence of (i.e., optionally combined with) an activator such as a tertiary or aromatic amine (e.g. dimethylaminopyridine, triethylamine, diisopropylethylamine, pyridine, and lutidine).

“Palladium catalysts” include, but are not limited to Pd(OAc)₂, Pd(dba)₂ (palladium dibenzylideneacetone), PdCl₂, (CH₃CN)₂PdCl₂, among others.

“Polar aprotic solvents” include but are not limited to dimethylacetamide (DMA), dimethylformamide (DMF), acetonitrile, or dimethyl sulfoxide (DMSO), preferably dimethyl sulfoxide.

“Approximately equimolar amounts” shall be used in the context of a reaction to describe amounts or quantities of reactants in the various reaction steps of the present invention wherein the ratio of reactants is approximately one to one. Such amounts in context may vary from about 2:1 to about 1:2, about 1.5:1 to about 1:1.5 or about 1.25:1 to about 1:1.25, or about 1:1.

The compounds identified herein can be named and numbered by using CS ChemDrawUltra 6.0® structure=name algorithm, or through conventions (e.g., CAS, IUPAC) which are accepted by those of ordinary skill in the art. The structures identified herein take precedence insofar as the identification of any compound is concerned.

The invention is illustrated firer in the following non-limiting examples.

### Example 1

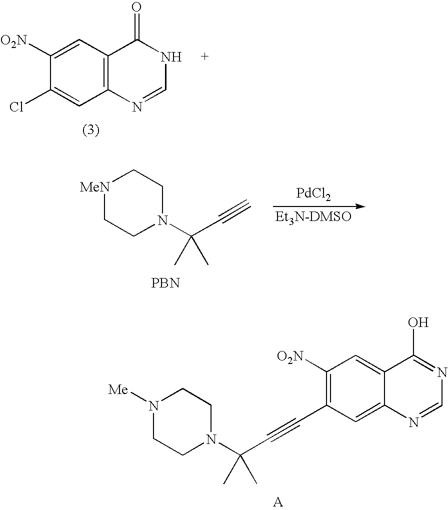

PBN (199.5 g, 1.2 mol, 1.2 equiv) (1,1-dimethyl-prop-2-ynyl)-4-methyl-piperazine) is dissolved in isopropyl acetate to a volume of 1 L. Compound 3 (226 g, 1 mol) (7-chloro-6-nitro-3H-quinazolin-4-one) and PdCl₂ (5.32 g, 0.03 mol, 3 mol %) are added to the solution, followed by Et₃N (2,024 g, 20 mol, 20 equiv) and dimethylsulfoxide (DMSO, 156.3 g, 2 mol). The resulting solution is heated to 85° C. for 6 h and then allowed to cool to ambient temperature. Water (5 L) is added, the mixture is agitated and then the phases separated. The organic phase is extracted twice with water (3 L) and the combined aqueous phases are extracted with isopropyl acetate (2 L) two times. Activated charcoal (25 g) is added to the combined organic phases, and the mixture is stirred 30 min and then filtered. The resulting solution is concentrated to dryness by rotary evaporation, and the residue is recrystallized from methanol/ethyl acetate to afford product A (249-284 g, 70-80% yield) (7-[3-methyl-3-(4-methyl-piperazin-1-yl)-but-1-ynyl]-6-nitro-quinazolin-4-ol) in >99% purity.

### Example 2

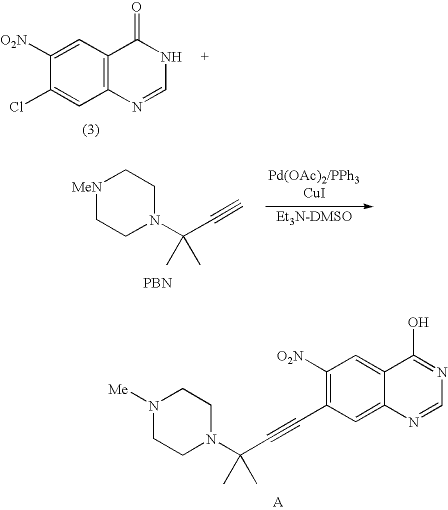

PBN (199.5 g, 1.2 mol, 1.2 equiv) is dissolved in isopropyl acetate to a volume of 1 L. Compound 3 (226 g, 1 mol), Pd(OAc)₂ (3.37 g, 0.015 mol, 1.5 mol %), and PPh₃ (7.87 g, 0.03 mol, 3 mol %) are added to the solution, followed by Et₃N (2,024 g, 3 mol, 3 equiv) and dimethylsulfoxide (DMSO, 2 L). The resulting solution is stirred at 25° C. for 4 h and then poured into an aqueous solution of N,N,N′,N′-tetramethylethylene diamine (TMEDA, 5% v/v). The mixture is extracted with isopropyl acetate (2 L) two times, and the combined organic phases are shed with another portion of aqueous TMEDA. The organic phase is separated and activated charcoal (25 g) is added. The mixture is stirred 30 min and filtered, and the resulting solution is concentrated to dryness by rotary evaporation, and the residue is recrystallized from methanol/ethyl acetate to afford product A (302-320 g, 85-90% yield) in >99% purity.

### Example 3

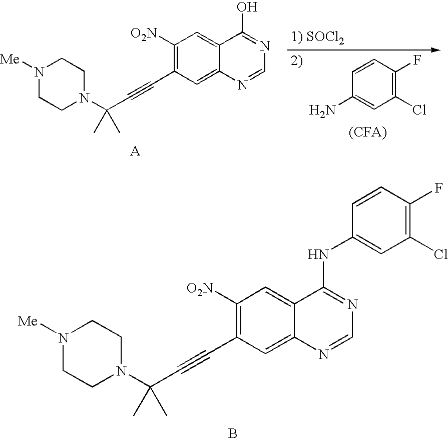

Compound A (355 g, 1 mol) is dissolved in toluene (2 L). Thionyl chloride (SOCl₂, 595 g, 5 mol, 5 equiv) and DMF (3.65 g, 0.05 mol, 0.05 equiv) are added, and the mixture is heated to reflux for 4 h. The resulting solution is cooled to 0° C., and 3-chloro fluoroaniline (CFA, 174.7 g, 1.2 mol. 1.2 equiv) is added as a 1 M solution in 2-methoxyethanol. The resulting mixture is stirred at 0° C. for 2 h, and then product B is purified by extraction with water (2 L), treatment of the organic phase with charcoal (20 g), filtration, and concentration to dryness. The residue is recrystallized from methanol/ethyl acetate to afford product B (386-410 g, 80-85% yield) (4-[N-3-chloro-4-fluorophenyl)]-7-[3-methyl-3-(4methyl-1-piperazinyl)-1-butynyl]-6-[nitro]quinazoline) in >99% purity.

### Example 4

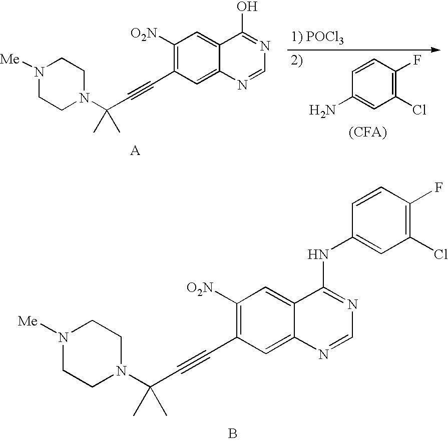

Compound A (355 g, 1 mol) is dissolved in toluene (2 L). Phosphoryl chloride (POCl₃, 230 g, 1.5 mol 1.5 equiv) and diisopropylethylamine (258.5 g, 2 mol, 2 equiv) are added, and the mixture is heated to 70° C. for 8 h. The resulting solution is cooled to 0° C., and 3-chloro-4-fluoroaniline (CFA, 174.7 g, 1.2 mol, 1.2 equiv) is added as a 1 M solution in isopropanol. The resulting mixture is stirred at 0° C. for 2 h, and then product B is purified by extraction with water (2 L), treatment of the organic phase with charcoal (20 g), filtration, and concentration to dryness. The residue is recrystallized from methanol/ethyl acetate to afford product B (362-381 g, 75-79% yield) in >99% purity.

### Example 5

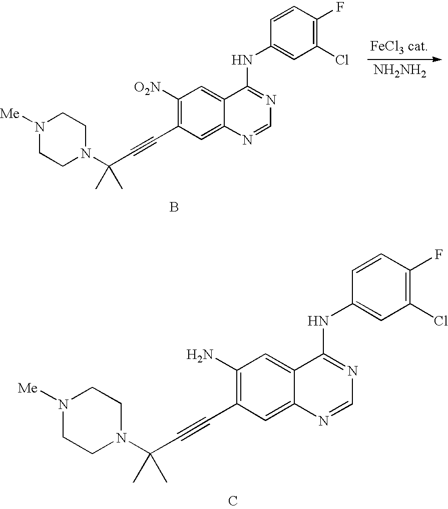

Compound B (482.2 g, 1 mol) is dissolved in methanol (3 L). Hydrazine (80%, 64.1 g, 2 mol, 2 equiv), FeCl₃ (1.62 g, 0.01 mol, 1 mol %) and activated charcoal (40 g) are added and the mixture is heated to reflux for 4 h. After the mixture is allowed to cool to ambient temperature, dichloromethane (1 L) is added the mixture is filtered and concentrated to dryness. The residue is purified by reslurrying with methanol and isolation by filtration. Product C (394-417 g, 87-92% yield) (4-[N-3-chloro-4-fluorophenyl)]-7-[3-methyl-3-(4-methyl-1-piperazinyl)-1-butynyl]-6-[amino]quinoline) is thus isolated in >99% purity.

### Example 6

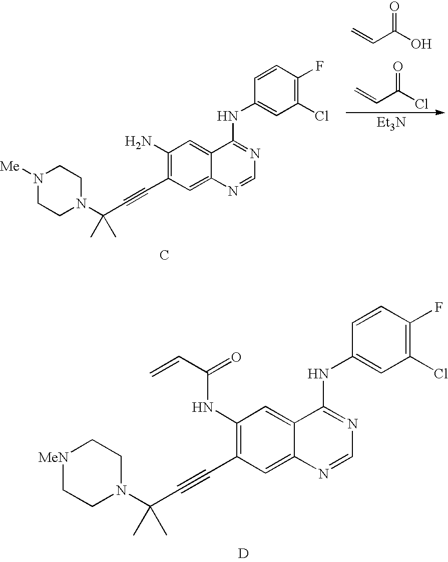

Compound C (453 g, 1 mol) is dissolved in THF (2 L) and the solution is cooled to 0° C. Acrylic acid (93.7 g, 1.3 mol, 1.3 equiv) and acryloyl chloride (108.6 g, 1.2 mol, 1.2 equiv) and triethylamine (253 g, 2.5 mol, 2.5 equiv) are added, and the resulting solution is stirred at 0° C. for 6 h and then allowed to warm to ambient temperature. Isolation and purification is carried out by solvent removal, trituration with isopropanol/water, and reslurrying acetonitrile. Compound D (324-350 g, 64-69% yield) (4-[N-3-chloro-4-fluorophenyl)]-7-[3-methyl-3-(4-methyl-1-piperazinyl)-1-butynyl]-6-[N-methyl acrylamide]quinoline is thus isolated in >99% purity.

### Example 7

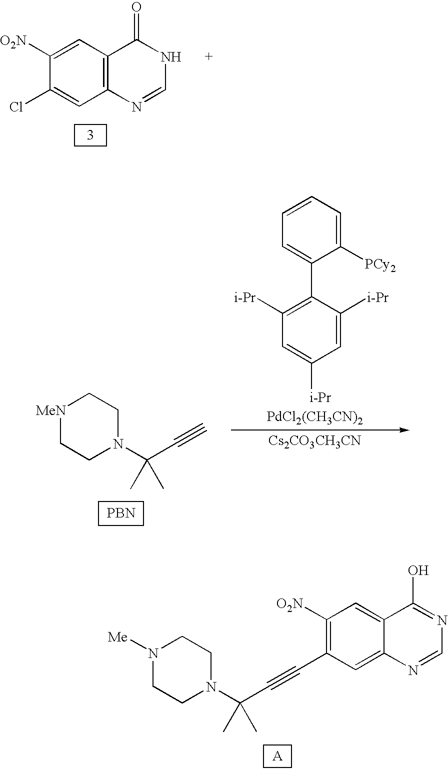

### Example 8

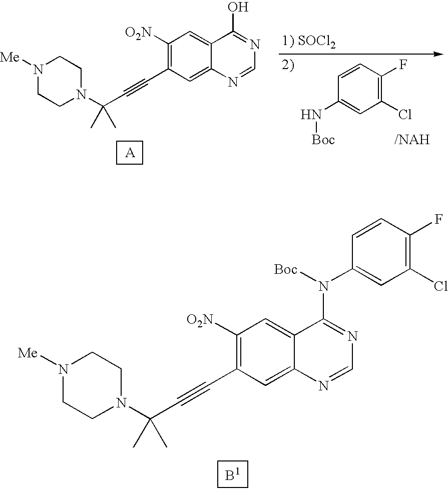

Compound A (355 g, 1 mol) is dissolved in toluene (2 L). Thionyl chloride (SOCl₂, 230 g, 1.5 mol, 1.5 equiv) and diisopropylethylamine (258.5 g, 2 mol, 2 equiv) are added, and the mixture is heated to 70° C. for 8 h. The resulting solution is cooled to 0° C., and Boc-3-chloro-4-fluoroaniline (Boc-CFA, 174.7 g, 1.2 mol, 1.2 equiv) is added as a 1 M solution in sodium hydroxide. The resulting mixture is stirred at 0° C. for 2 h, and than product B¹ is purified by extraction with water (2 L), treatment of the organic phase with charcoal (20 g), filtration, and concentration to dryness. The residue is recrystallized from methanol/ethyl acetate to afford product B¹ (362-381 g, 75-79% yield) in >99% purity.

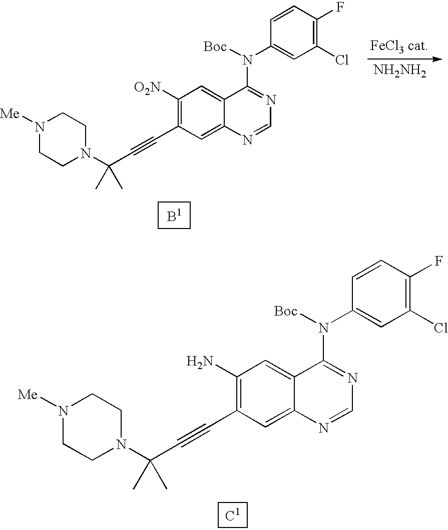

Compound B¹ (482.2 g, 1 mol) is dissolved in methanol (3 L). Hydrazine (80%, 64.1 g, 2 mol, 2 equiv), FeCl₃ (1.62 g, 0.01 mol, 1 mol %) and activated charcoal (40 g) are added and mixture is heated to reflux for 4 h. After the mixture is allowed to cool to ambient temperature, dichloromethane (1 L) is added the mixture is filtered and concentrated to dryness. The residue is purified by reslurrying with methanol and isolation by filtration. Product C¹ (394-417 g, 87-92% yield) (4-[N-Boc-3-chloro-4-fluorophenyl)]-7-[3-methyl-3-(4-methyl-1-piperazinyl)-1-butynyl]-6-[amino]quinazoline) is tis isolated in >99% purity.

### Example 10

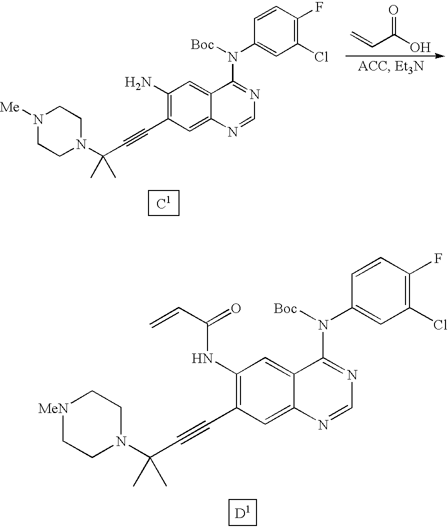

Compound C¹ (453 g, 1 mol) is dissolved in THF (2 L) and the solution is cooled to 0° C. Acrylic acid (93.7 g, 1.3 mol. 1.3 equiv) and acryloyl chloride (108.6 g, 1.2 mol. 1.2 equiv) and triethylamine (253 g, 2.5 mol, 2.5 equiv) are added, and the resulting solution is stirred at 0° C. for 6 h and hen allowed to warm to ambient temperature. Isolation and purification is carried out by solvent removal, trituration with, and reslurrying acetonitrile. Compound D¹ (324-350 g, 64-69% yield) (4-[N-Boc-3-chloro-4-fluorophenyl)]-7-[3-methyl-3-(4-methyl-1-piperazinyl)-1-butynyl]-6-[N-methyl acrylamide]quinazoline is thus isolated in >99% purity. The acid-labile protecting group BOC is removed in near quantitative yield using an organic acid such as toluenesulfonic acid (TsOH) or methanesulfonic acid in methanol to provide compound D (see example 6).

1. A process for synthesizing 7-alkynyl-4-aminoquinazoline compounds having the formula (1): 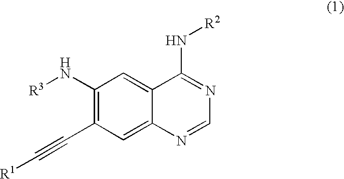
in which R¹ is: 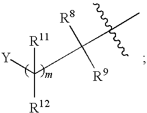
where (i) R⁸ and R⁹ are each independently a hydrogen atom, or (ii) R⁸ and R⁹ are each independently a C₁-C₅ alkyl group optionally substituted by a C₁-C₅ alkoxy group, m is an integer of 0-3, R¹¹ and R¹² are each independently a hydrogen atom or a C₁-C₅ alkyl group, and Y is a hydrogen atom, a hydroxyl group, a C₁-C₅ alkoxy group, a C₁-C₅ alkanoyloxy group, 4-(C₁-C₅ alkyl)piperazin-1-yl, di(C₁-C₅ alkyl)amino, —N(R¹⁶)—(CO)ᵤ—(CR¹⁷R¹⁸)ᵥ—(CO)ⱼ—R¹⁹ wherein R¹⁶ is a hydrogen atom, or a C₁-C₅ alkyl group optionally substituted by a cyano group or a C₁-C₅ alkoxy group, R¹⁷ and R¹⁸ are each independently a hydrogen atom or a C₁-C₅ alkyl group, u and j are each 0 or 1, v is an integer of 1-5 and R¹⁹ is a hydrogen atom, a hydroxyl group, a cyano group, an amino group, a C₁-C₅ alkoxy group, a morpholino group, 4-(C₁-C₅ alkyl)piperazin-1-yl or di (C₁-C₅ alkyl) amino;
in which R³ is: 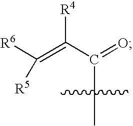
and R⁴, R⁵, and R⁶ are each independently a hydrogen atom, a halogen atom or a C₁-C₅ alkyl group optionally substituted by a halogen atom, a morpholino group, 4-(C₁-C₅ alkyl) piperazin-1-yl or di(C₁-C₅ alkyl)amino; and
in which R² is: 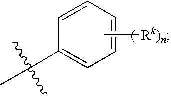
where n is an integer of 0-3 and R^{k} is a hydrogen atom, a halogen atom, a hydroxyl group, a cyano group, a nitro group, a trifluoromethyl group, a C₁-C₅ alkyl group, a C₁-C₅ alkoxy group, —S(O)_{f}R¹³ (wherein f is an integer of 0-2 and R¹³ is a C₁-C₅ alkyl group), —NR¹⁴R¹⁵ (wherein R¹⁴ and R¹⁵ are each independently a hydrogen atom, a C₁-C₅ alkyl group, a C₁-C₅ alkanoyl group, or a C₁-C₅ alkylsulfonyl group, a C₁-C₅ alkenyl group, a C₁-C₅ alkynyl group, or a C₁-C₅ alkanoyl group, the process comprising:
(a) derivatizing 7-chloro-6-nitro-3H-quinazolin-4-one at the 7-position with a moiety of formula (IA): 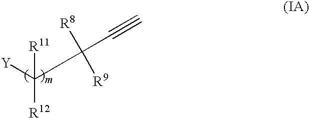
where R⁸, R⁹, R¹¹, R¹², Y, and m are as defined above, by reacting approximately equimolar amounts of 7-chloro-6-nitro-3H-quinazolin-4-one and a compound of formula (IA) in a reaction medium comprising a polar aprotic solvent, a palladium catalyst, and optionally an amine acid scavenger, to form a first reaction product;
(b) reacting approximately equimolar amounts of the first reaction product and a compound of the formula (NH₂)—R² in a reaction medium comprising a halogenating agent to form a derivatized 7-alkynyl-4-aminoquinazoline as a second reaction product;
(c) reacting the second reaction product under reducing conditions with a reducing agent, thereby forming a third reaction product; and
(d) reacting the third reaction product with an approximately equimolar amount of an acylating agent.

2. The process of claim 1, wherein 7-chloro-6-nitro-3H-quinazolin-4-one is derivatized at the 7-position with a moiety of formula (IA) through a Sonogashira coupling reaction between equimolar amounts of 7-chloro-6-nitro-3H-quinazolin-4-one and a compound of formula (IA), wherein the Sonogashira coupling reaction occurs at a temperature of between about 20° C. to about 100° C. in a reaction medium comprising an amine acid scavenger, an aprotic polar solvent, and a palladium catalyst and said reducing conditions occur using a reducing agent and a Lewis acid at an approximately 2:1 molar ratio of reducing agent:second reaction product.

3. The process of claim 1, wherein:
(a) the halogenating agent is selected from the group consisting of a thionyl halide, phosphorous oxychloride, and phosphorous halide;
(b) the reducing agent is selected from the group consisting of hydrazine, hydrogen gas, phenylhydrazine, Sn/HCl, SnCl₂/HCl, Zn/H₂O, NaBH₄/CuCl, NaBH₄/TiCl₄, Fe, and Na₂S;
(c) the acylating agent is selected from the group consisting of (i) acylating agents derived by the combination of a carboxylic acid and an activating agent, and (ii) carboxylic acid derivatives,
(d) the polar aprotic solvent is dimethylacetamide (DMA), dimethylformamide (DMF), acetonitrile or dimethylsulfoxide (DMSO); and
(e) the amine acid scavenger is triethylamine.

4. The process of claim 3, wherein:
(a) the halogenating agent is thionyl chloride;
(b) the reducing agent is hydrazine;
(c) the Lewis acid is FeCl₃; and
(d) the acylating agent is derived from the combination of acrylic acid and acryloyl chloride (ACC).

5. A process for synthesizing 4-[N-3-chloro-4-fluorophenyl)]-7-[3-methyl-3-(4-methyl-1-piperazinyl)-1-butynyl]-6-[N-methyl acrylamide]quinazoline, the process comprising:
(a) reacting approximately equimolar amounts of 7-[3-methyl-3-(4-methyl-piperazin-1-yl)-but-1-ynyl]-6-nitro-quinazolin-4-ol and 3-chloro-4-fluoro-phenylamine
(i) at a temperature of between about 0° C. to about 90° C., and
(ii) in a reaction medium comprising an halogenating agent to form 4-[N-3-chloro-4-fluorophenyl)]-7-[3-methyl-3-(4-methyl-1-piperazinyl)-1-butynyl]-6-[nitro]quinazoline;
(b) reacting 4-[N-3-chloro-4-fluorophenyl)]-7-[3-methyl-3-(4-methyl-1-piperazinyl)-1-butynyl]-6-[nitro]quinazoline with a reducing agent
(i) at an approximately 2:1 molar ratio of reducing agent: 4-[N-3-chloro-4-fluorophenyl)]-7-[3-methyl-3-(4-methyl-1-piperazinyl)-1-butynyl]-6-[nitro]quinazoline,
(ii) at a temperature of between about 20° C. to about 90° C., and
(iii) in a reaction medium comprising a Lewis acid, thereby forming 4-[N-3-chloro-4-fluorophenyl)]-7-[3-methyl-3-(4-methyl-1-piperazinyl)-1-butynyl]-6-[amino]quinazoline; and
(c) reacting approximately equimolar amounts of 4-[N-3-chloro-4-fluorophenyl)]-7-[3-methyl-3-(4-methyl-1-piperazinyl)-1-butynyl]-6-[amino]quinazoline with an acylating agent at a temperature of between about 0° C. to about 40° C., in a reaction medium comprising an amine acid scavenger.

6. The process of claim 5, wherein:
(a) the halogenating agent is selected from the group consisting of a thionyl halide, phosphorous oxychloride, and phosphorous halide;
(b) the reducing agent is selected from the group consisting of hydrazine, hydrogen gas, phenylhydrazine, Sn/HCl, SnCl₂/HCl, Zn/H₂O, NaBH₄/CuCl, NaBH₄/TiCl₄, Fe, and Na₂S;
(c) the acylating agent is selected from the group consisting of (i) acylating agents derived by the combination of a carboxylic acid and an activating agent, and (ii) carboxylic acid derivatives; and
(d) the amine acid scavenger is triethylamine.

7. The process of claim 5, wherein:
(a) the halogenating agent is thionyl chloride;
(b) the reducing agent is hydrazine;
(c) the Lewis acid is FeCl₃;
(d) the acylating agent is obtained from a combination of acrylic acid and acryloyl chloride (ACC); and
(e) the amine acid scavenger is triethylamine

8. The process of claim 5, wherein 7-[3-methyl-3-(4-methyl-piperazin-1-yl)-but-1-ynyl]-6-nitro-quinazolin-4-ol is synthesized by a Sonogashira coupling reaction between 7-chloro-6-nitro-3H-quinazolin-4-one and 1-(1,1-dimethyl-prop-2-ynyl)-4-methyl-piperazine.

9. The process of claim 8, wherein the Sonogashira coupling reaction occurs in a reaction medium comprising an amine acid scavenger, a polar aprotic solvent, and a palladium catalyst.

10. A process for synthesizing 4-[N-3-chloro-4-fluorophenyl)]-7-[3-methyl-3-(4-methyl-1-piperazinyl)-1-butynyl]-6-[N-methyl acrylamide]quinazoline, the process comprising:
(a) reacting approximately equimolar amounts of 7-chloro-6-nitro-3H-quinazolin-4-one and 1-(1,1-dimethyl-prop-2-ynyl)-4-methyl-piperazine
(i) at a temperature of between about 10° C. to about 90° C., and
(ii) in a reaction medium comprising an amine acid scavenger, a polar aprotic solvent, and a palladium catalyst, thereby forming 7-[3-methyl-3-(4-methyl-piperazin-1-yl)-but-1-ynyl]-6-nitro-quinazolin-4-ol;
(b) reacting approximately equimolar amounts of 7-[3-methyl-3-(4-methyl-piperazin-1-yl)-but-1-ynyl]-6-nitro-quinazolin-4-ol with 3-chloro-4-fluoro-phenylamine
(i) at a temperature of between about 0° C. to about 90° C., and
(ii) in a reaction medium comprising a halogenating agent thereby forming 4-[N-3-chloro-4-fluorophenyl)]-7-[3-methyl-3-(4-methyl-1-piperazinyl)-1-butynyl]-6-[nitro]quinazoline;
(c) reacting 4-[N-3-chloro-4-fluorophenyl)]-7-[3-methyl-3-(4-methyl-1-piperazinyl)-1-butynyl]-6-[nitro]quinazoline with a reducing agent
(i) at an approximately 2:1 molar ratio of reducing agent: 4-[N-3-chloro-4-fluorophenyl)]-7-[3-methyl-3-(4-methyl-1-piperazinyl)-1-butynyl]-6-[nitro]quinazoline,
(ii) at a temperature of between about 20° C. to about 90° C., and
(iii) in a reaction medium comprising a Lewis acid, thereby forming 4-[N-3-chloro-4-fluorophenyl)]-7-[3-methyl-3-(4-methyl-1-piperazinyl)-1-butynyl]-6-[amino]quinazoline; and
(d) reacting approximately equimolar amounts of 4-[N-3-chloro-4-fluorophenyl)]-7-[3-methyl-3-(4-methyl-1-piperazinyl)-1-butynyl]-6-[amino]quinazoline with an acylating agent at a temperature of between about 0C to about 40° C., in a reaction medium optionally comprising an amine acid scavenger.

11. The process of claim 10, wherein:
(a) the polar aprotic solvent is selected from the group consisting of dimethylacetamide (DMA), dimethylformamide (DMF), acetonitrile, and dimethyl sulfoxide (DMSO);
(b) the halogenating agent is selected from the group consisting of a thionyl halide, phosphorous oxychloride, and phosphorous halide;
(c) the reducing agent is selected from the group consisting of hydrazine, Zn/water, Zn/HCl, Zn/NaOH, Zn/NH₃, Fe, Fe/HOAC, Fe/HCl, Sn/HCl, SnCl₂/HCl, Na₂S, Na₂S₂, Na₂S₂O₄, (NH₄)₂S, NaBH₄/CuCl, Al₂Te₃/H₂, PhNHNH₂ (phenylhydrazine), NaO₂CH/KH₂PO₄, CO/H₂O/Se/Et₃N, Fe(CO)₅, Fe₃(CO)₁₂/Al₂O₃, hydrogen gas and NaBH₄/TiCl₄; and
(d) the acylating agent is selected from the group consisting of (i) acylating agents derived by the combination of a carboxylic acid and an activating agent, and (ii) carboxylic acid derivatives.

12. The process of claim 10, wherein:
(a) the polar aprotic solvent is dimethyl sulfoxide (DMSO);
(b) the halogenating agent is thionyl chloride;
(c) the reducing agent is hydrazine;
(d) the Lewis acid is FeCl₃; and
(e) the acylating agent is derived by the combination of acrylic acid and acryloyl chloride (ACC).

13. A process for derivatizing 7-chloro-6-nitro-3H-quinazolin-4-one at the 7-position with a moiety of formula (IA): 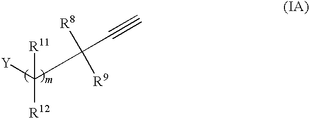
where (i) R⁸ and R⁹ are each independently a hydrogen atom, or (ii) R⁸ and R⁹ are each independently a C₁-C₅ alkyl group optionally substituted by a C₁-C₅ alkoxy group, m is an integer of 0-3, R¹¹ and R¹² are each independently a hydrogen atom or a C₁-C₅ alkyl group, and Y is a hydrogen atom, a hydroxyl group, a C₁-C₅ alkoxy group, a C₁-C₅ alkanoyloxy group, 4-C₁-C₅ alkylpiperazin-1-yl, di(C₁-C₅ alkyl)amino, —N(R¹⁶)—(CO)ᵤ—(CR¹⁷R¹⁸)ᵥ—(CO)ⱼ—R¹⁹ (wherein R¹⁶ is a hydrogen atom, or a C₁-C₅ alkyl group optionally substituted by a cyano group or a C₁-C₅ alkoxy group, R¹⁷ and R¹⁸ are each independently a hydrogen atom or a C₁-C₅ alkyl group, u and j are each 0 or 1, v is an integer of 1-5 and R¹⁹ is a hydrogen atom, a hydroxyl group, a cyano group, an amino group, a C₁-C₅ alkoxy group, a morpholino group, 4-C₁-C₅ alkylpiperazin-1-yl or di(C₁-C₅ alkyl) amino, the process comprising reacting approximately equimolar amounts of 7-chloro-6-nitro-3H-quinazolin-4-one and a compound of formula (IA) at a temperature of between about 20° C. to about 100° C. in a reaction medium comprising an amine acid scavenger, a polar aprotic solvent, and a palladium catalyst.

14. The process of claim 13, wherein 7-chloro-6-nitro-3H-quinazolin-4-one is derivatized at the 7-position with a moiety of formula (IA) by a Sonogashira coupling reaction between 7-chloro-6-nitro-3H-quinazolin-4-one and a compound of formula (IA).

15. The process of claim 14, wherein the Sonogashira coupling reaction occurs in a reaction medium comprising triethyl amine, dimethyl sulfoxide, and a palladium chloride catalyst.

16. A process for synthesizing a compound of the formula: 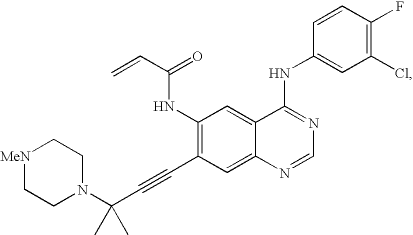
the process comprising:
(a) derivatizing a compound of formula (I) 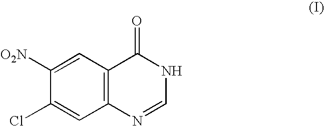
at the 7-position with a compound of formula (II) 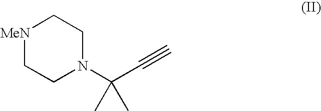
by reacting approximately equimolar amounts of the compounds of formulae (I) and (II) in a reaction medium comprising an amine acid scavenger, a polar aprotic solvent, and a palladium catalyst to yield a compound of formula (III) 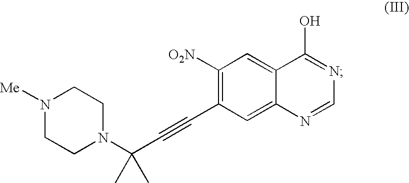
(b) reacting approximately equimolar amounts of the compound of formula (III) and 3-chloro-4-fluoroaniline in a reaction medium comprising a halogenating agent, thereby forming a compound of formula (IV) 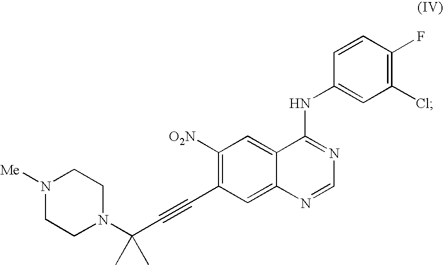
(c) reacting the compound of formula (IV) with a reducing agent at an approximately 2:1 molar ratio of reducing agent: compound of formula (IV) in a reaction medium optionally comprising a Lewis acid, thereby forming a compound of formula (V) 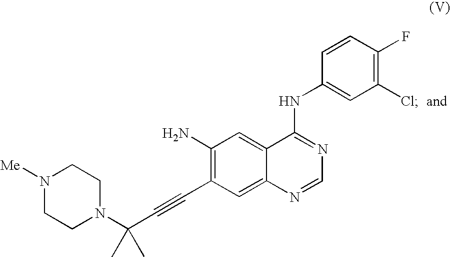
(d) reacting approximately equimolar amounts of the compound of formula (V) with an acylating agent in a reaction medium comprising an amine acid scavenger.

17. The process of claim 16, wherein:
(a) the polar aprotic solvent is selected from the group consisting of dimethylacetamide (DMA), dimethylformamide (DMF), acetonitrile, and dimethyl sulfoxide (DMSO);
(b) the halogenating agent is selected from the group consisting of a thionyl halide, phosphorous oxychloride, and phosphorous halide;
(c) the reducing agent is selected from the group consisting of hydrazine, Zn/water, Zn/HCl, Zn/NaOH, Zn/NH₃, Fe, Fe/HOAC, Fe/HCl, Sn/HCl, SnCl₂/HCl, Na₂S, Na₂S₂, Na₂S₂O₄, (NH₄)₂S, NaBH₄/CuCl, Al₂Te₃/H₂, PhNHNH₂ (phenylhydrazine), NaO₂CH/KH₂PO₄, CO/H₂O/Se/Et₃N, Fe(CO)₅, Fe₃(CO)₁₂/Al₂O₃, hydrogen gas, NaH, KH, LiH and NaBH₄/TiCl₄;
(d) the acylating agent is selected from the group consisting of (i) acylating agents derived by the combination of a carboxylic acid and an activating agent, and (ii) carboxylic acid derivatives; and
(e) the amine acid scavenger is triethylamine.

18. The process of claim 16, wherein:
(a) the polar aprotic solvent is dimethyl sulfoxide (DMSO);
(b) the halogenating agent is thionyl chloride;
(c) the reducing agent is hydrazine;
(d) the Lewis acid is FeCl₃; and
(e) the acylating agent is derived by the combination of acrylic acid and acryloyl chloride (ACC).

19. A process for synthesizing a compound of the formula: 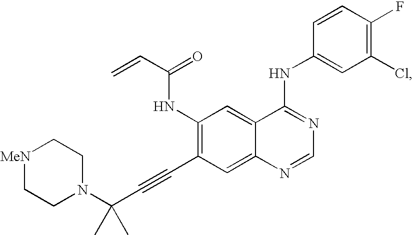
the process comprising:
(a) reacting approximately equimolar amounts of the compound of formula (III) 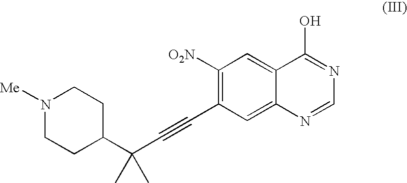
and 3-chloro-4-fluoroaniline in a reaction medium comprising a halogenating agent, thereby forming a compound of formula (IV) 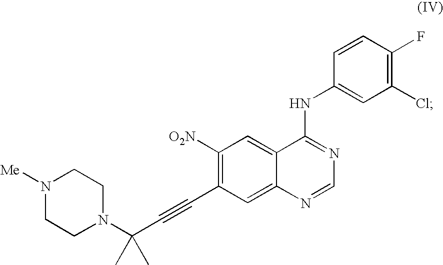
(b) reacting the compound of formula (IV) with a reducing agent at an approximately 2:1 molar ratio of reducing agent: compound of formula (IV) in a reaction medium optionally comprising a Lewis acid, thereby forming a compound of formula (V) 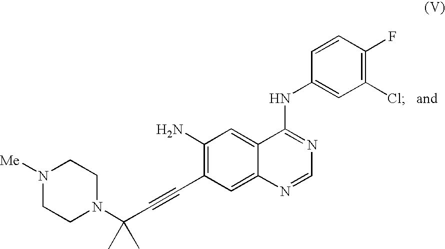
(c) reacting approximately equimolar amounts of the compound of formula (V) with an acylating agent in a reaction medium comprising an amine acid scavenger.

20. The process of claim 19, wherein:
(a) the halogenating agent is selected from the group consisting of a thionyl halide, phosphorous oxychloride, and phosphorous halides;
(b) the reducing agent is selected from the group consisting of hydrazine, Zn/water, Zn/HCl, Zn/NaOH, Zn/NH₃, Fe, Fe/HOAC, Fe/HCl, Sn/HCl, SnCl₂/HCl, Na₂S, Na₂S₂, Na₂S₂O₄, (NH₄)₂S, NaBH₄/CuCl, Al₂Te₃/H₂, PhNHNH₂ (phenylhydrazine), NaO₂CH/KH₂PO₄, CO/H₂O/Se/Et₃N, Fe(CO)₅, Fe₃(CO)₁₂/Al₂O₃, hydrogen gas, NaH, KH, LiH and NaBH₄/TiCl₄;
(c) the acylating agent is selected from the group consisting of (i) acylating agents derived by the combination of a carboxylic acid and an activating agent, and (ii) carboxylic acid derivatives; and
(d) the amine acid scavenger is triethylamine.

21. The process of claim 19, wherein:
(a) the halogenating agent is thionyl chloride;
(b) the reducing agent is hydrazine;
(c) the Lewis acid is FeCl₃; and
(d) the acylating agent is derived by the combination of acrylic acid and acryloyl chloride (ACC).

22. A process for synthesizing a compound of the formula: 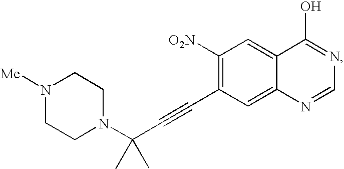
the process comprising derivatizing a compound of formula (I) 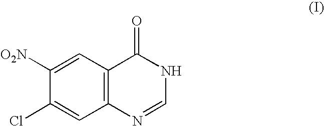
at the 7-position with a compound of formula (II) 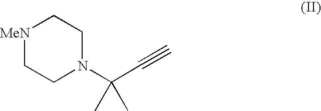
by reacting approximately equimolar amounts of the compounds of formulae (I) and (II) in a reaction medium comprising an amine acid scavenger, a polar aprotic solvent, and a palladium catalyst

23. A compound of the formula:

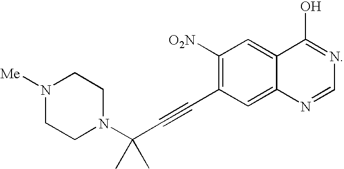

24. A process for synthesizing 4-[N-3-chloro-4-fluorophenyl)]-7-[3-methyl-3-(4-methyl-1-piperazinyl)-1-butynyl]-6-[N-methyl acrylamide]quinazoline, the process comprising:
(a) reacting approximately equimolar amounts of 7-[3-methyl-3-(4-methyl-piperazin-1-yl)-but-1-ynyl]-6-nitro-quinazolin-4-ol and N-protected 3-chloro-4-fluoro-phenylamine
(i) at a temperature of between about 0° C. to about 90° C., and
(ii) in a reaction medium comprising an halogenating agent and a base to form 4-[N-protected-3-chloro-4-fluorophenyl)]-7-[3-methyl-3-(4-methyl-1-piperazinyl)-1-butynyl]-6-[nitro]quinazoline;
(b) reacting 4-[N-protected-3-chloro-4-fluorophenyl)]-7-[3-methyl-3-(4-methyl-1-piperazinyl)-1-butynyl]-6-[nitro]quinazoline with a reducing agent
(i) at an approximately 2:1 molar ratio of reducing agent: 4-[N-protected-3-chloro-4-fluorophenyl)]-7-[3-methyl-3-(4-methyl-1-piperazinyl)-1-butynyl]-6-[nitro]quinazoline,
(ii) at a temperature of between about 20° C. to about 90° C., in a reaction medium optionally comprising a Lewis acid, thereby forming 4-[N-protected-3-chloro-4-fluorophenyl)]-7-[3-methyl-3-(4-methyl-1-piperazinyl)-1-butynyl]-6-[amino]quinazoline; and
(c) reacting approximately equimolar amounts of 4-[N-protected-3-chloro-4-fluorophenyl)]-7-[3-methyl-3-(4-methyl-1-piperazinyl)-1-butynyl]-6-[amino]quinazoline with an acylating agent at a temperature of between about 0° C. to about 40° C., and in a reaction medium comprising an amine acid scavenger.

25. The process of claim 24, wherein:
(a) the N-protected 3-chloro-4-fluoro-phenylamine is N-Boc-3-chloro-4-fluoro-phenylamine;
(b) the halogenating agent is selected from the group consisting of a thionyl halide, phosphorous oxychloride, and phosphorous halide;
(c) the reducing agent is selected from the group consisting of hydrazine, Zn/water, Zn/HCl, Zn/NaOH, Zn/NH₃, Fe, Fe/HOAC, Fe/HCl, Sn/HCl, SnCl₂/HCl, Na₂S, Na₂S₂, Na₂S₂O₄, (NH₄)₂S, NaBH₄/CuCl, Al₂Te₃/H₂, PhNHNH₂ (phenylhydrazine), NaO₂CH/KH₂PO₄, CO/H₂O/Se/Et₃N, Fe(CO)₅, Fe₃(CO)₁₂/Al₂O₃, hydrogen gas and NaBH₄/TiCl₄, NaH, LiH, and KH;
(d) the acylating agent is selected from the group consisting of (i) acylating agents derived by the combination of a carboxylic acid and an activating agent, and (ii) carboxylic acid derivatives; and
(e) the amine acid scavenger is triethylamine.

26. The process of claim 24, wherein:
(a) the N-protected 3-chloro-4-fluoro-phenylamine is N-Boc-3-chloro-4-fluoro-phenylamine;
(b) the halogenating agent is thionyl chloride;
(c) the reducing agent is hydrazine;
(d) the Lewis acid is FeCl₃;
(e) the acylating agent is derived by the combination of acrylic acid and acryloyl chloride (ACC); and
(f) the amine acid scavenger is triethylamine.

27. The process of claim 24, wherein 7-[3-methyl-3-(4-methyl-piperazin-1-yl)-but-1-ynyl]-6-nitro-quinazolin-4-ol is synthesized by a Sonogashira coupling reaction between 7-chloro-6-nitro-3H-quinazolin-4-one and 1-(1,1-dimethyl-prop-2-ynyl)-4-methyl-piperazine.

28. The process of claim 27, wherein the Sonogashira coupling reaction occurs in a reaction medium comprising an amine acid scavenger, a polar aprotic solvent, and a palladium catalyst.

29. A process for synthesizing 4-[N-3-chloro-4-fluorophenyl)]-7-[3-methyl-3-(4-methyl-1-piperazinyl)-1-butynyl]-6-[N-methyl acrylamide]quinazoline, the process comprising:
(a) reacting approximately equimolar amounts of 7-chloro-6-nitro-3H-quinazolin-4-one and 1-(1,1-dimethyl-prop-2-ynyl)-4-methyl-piperazine
(i) at a temperature of between about 10° C. to about 90° C., and
(ii) in a reaction medium comprising an amine acid scavenger, a polar aprotic solvent, and a palladium catalyst, thereby forming 7-[3-methyl-3-(4-methyl-piperazin-1-yl)-but-1-ynyl]-6-nitro-quinazolin-4-ol;
(b) reacting approximately equimolar amounts of 7-[3-methyl-3-(4-methyl-piperazin-1-yl)-but-1-ynyl]-6-nitro-quinazolin-4-ol with Boc-3-chloro-4-fluoro-phenylamine
(i) at a temperature of between about 0° C. to about 90° C., and
(ii) in a reaction medium comprising a halogenating agent thereby forming 4-[N-Boc-3-chloro-4-fluorophenyl)]-7-[3-methyl-3-(4-methyl-1-piperazinyl)-1-butynyl]-6-[nitro]quinazoline;
(c) reacting 4-[N-Boc-3-chloro-4-fluorophenyl)]-7-[3-methyl-3-(4-methyl-1-piperazinyl)-1-butynyl]-6-[nitro]quinazoline with a reducing agent
(i) at an approximately 2:1 molar ratio of reducing agent: 4-[N-Boc-3-chloro-4-fluorophenyl)]-7-[3-methyl-3-(4-methyl-1-piperazinyl)-1-butynyl]-6-[nitro]quinazoline,
(ii) at a temperature of between about 20° C. to about 90° C., and
(iii) in a reaction medium optionally comprising a Lewis acid, thereby forming 4-[N-Boc-3-chloro-4-fluorophenyl)]-7-[3-methyl-3-(4-methyl-1-piperazinyl)-1-butynyl]-6-[amino]quinazoline; and
(d) reacting approximately equimolar amounts of 4-[N-Boc-3-chloro-4-fluorophenyl)]-7-[3-methyl-3-(4-methyl-1-piperazinyl)-1-butynyl]-6-[amino]quinazoline with an acylating agent at a temperature of between about 0° C. to about 40° C., and in a reaction medium comprising an amine acid scavenger.

30. A process for synthesizing a compound of the formula: 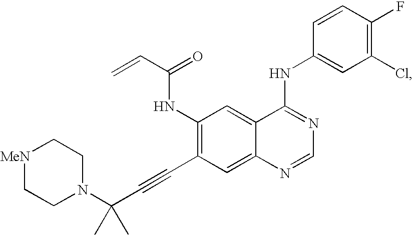
the process comprising:
(a) derivatizing a compound of formula (I) 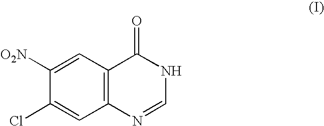
at the 7-position with a compound of formula (II) 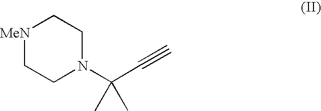
by reacting approximately equimolar amounts of the compounds of formulae (I) and (II) in a reaction medium comprising an amine acid scavenger, a polar aprotic solvent, and a palladium catalyst to yield a compound of formula (III) 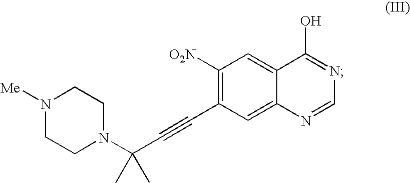
(b) reacting approximately equimolar amounts of the compound of formula (III) and Boc-3-chloro-4-fluoroaniline in a reaction medium comprising a halogenating agent, thereby forming a compound of formula (IV) 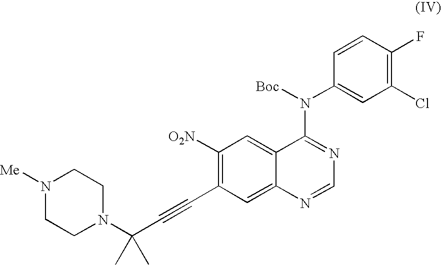
(c) reacting the compound of formula (IV) with a reducing agent at an approximately 2:1 molar ratio of hydrazine: compound of formula (IV) in a reaction medium comprising a Lewis acid, thereby forming a compound of formula (V) 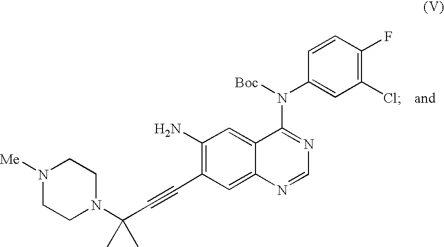
(d) reacting approximately equimolar amounts of the compound of formula (V) with an acylating agent in a reaction medium comprising an amine acid scavenger.

31. The process of claim 30, wherein:
(a) the polar aprotic solvent is selected from the group consisting of dimethylacetamide (DMA), dimethylformamide (DMF), acetonitrile, and dimethyl sulfoxide (DMSO);
(b) the halogenating agent is selected from the group consisting of a thionyl halide, phosphorous oxychloride, and phosphorous halide;
(c) the reducing agent is selected from the group consisting of hydrazine, Zn/water, Zn/HCl, Zn/NaOH, Zn/NH₃, Fe, Fe/HOAC, Fe/HCl, Sn/HCl, SnCl₂/HCl, Na₂S, Na₂S₂, Na₂S₂O₄, (NH₄)₂S, NaBH₄/CuCl, Al₂Te₃/H₂, PhNHNH₂ (phenylhydrazine), NaO₂CH/KH₂PO₄, CO/H₂O/Se/Et₃N, Fe(CO)₅, Fe₃(CO)₁₂/Al₂O₃, hydrogen gas, NaH, KH, LiH and NaBH₄/TiCl₄;
(d) the acylating agent is selected from the group consisting of (i) acylating agents derived by the combination of a carboxylic acid and an activating agent, and (ii) carboxylic acid derivatives; and
(e) the amine acid scavenger is triethylamine.

32. The process of claim 31, wherein:
(a) the polar aprotic solvent is dimethyl sulfoxide (DMSO);
(b) the halogenating agent is thionyl chloride;
(c) the reducing agent is hydrazine;
(d) the Lewis acid is FeCl₃;
(e) the acylating agent is derived by the combination of acrylic acid and acryloyl chloride (ACC); and
(f) the amine acid scavenger is triethylamine.

33. A process for synthesizing a compound of the formula: 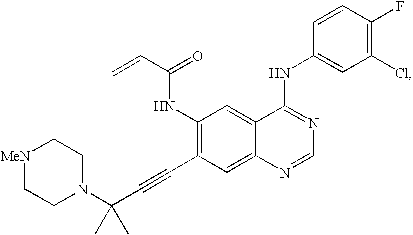
the process comprising:
(a) reacting approximately equimolar amounts of the compound of formula (III) 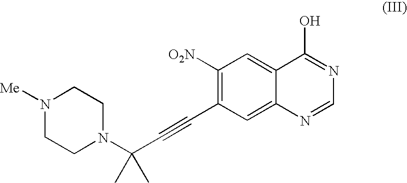
and Boc-3-chloro-4-fluoroaniline in a reaction medium comprising a halogenating agent, thereby forming a compound of formula (IV) 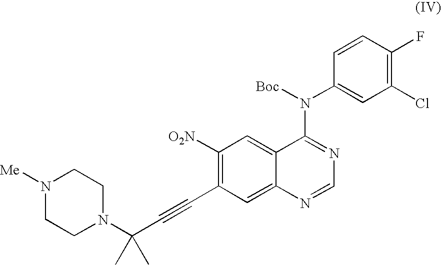
(b) reacting the compound of formula (IV) with a reducing agent at an approximately 2:1 molar ratio of reducing agent: compound of formula (IV) in a reaction medium optionally comprising a Lewis acid, thereby forming a compound of formula (V) 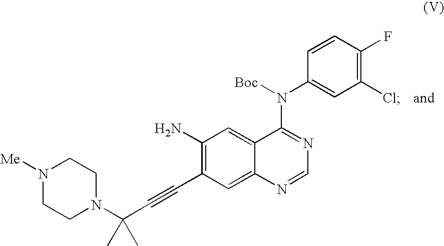
(c) reacting approximately equimolar amounts of the compound of formula (V) with an acylating agent in a reaction medium comprising an amine acid scavenger.

34. The process of claim 33, wherein:
(a) the halogenating agent is selected from the group consisting of a thionyl halide, phosphorous oxychloride, and phosphorous halides;
(b) the reducing agent is selected from the group consisting of hydrazine, Zn/water, Zn/HCl, Zn/NaOH, Zn/NH₃, Fe, Fe/HOAC, Fe/HCl, SnMCl, SnCl₂/HCl, Na₂S, Na₂S₂, Na₂S₂O₄, (NH₄)₂S, NaBH₄/CuCl, Al₂Te₃/H₂, PhNHNH₂ (phenylhydrazine), NaO₂CH/KH₂PO₄, CO/H₂O/Se/Et₃N, Fe(CO)₅, Fe₃(CO)₁₂/Al₂O₃, hydrogen gas, NaH, KH, LiH and NaBH₄/TiCl₄;
(c) the acylating agent is selected from the group consisting of (i) acylating agents derived by the combination of a carboxylic acid and an activating agent, and (ii) carboxylic acid derivatives; and
(d) the amine acid scavenger is triethylamine.

35. The process of claim 33, wherein:
(a) the halogenating agent is thionyl chloride;
(b) the reducing agent is hydrazine;
(c) the Lewis acid is FeCl₃;
(d) the acylating agent is derived by the combination of acrylic acid and acryloyl chloride (ACC); and
(e) the amine acid scavenger is triethylamine.
